(19) United States
(12) Patent Application Publication
(10) Pub. No.: US 2026/0171220 A1
(43) Pub. Date: Jun. 18, 2026

(54) AI-ENABLED EARLY DETECTION AND PREVENTION OF ADVERSE HEALTH OUTCOMES

(71) Applicants: Morteza Naghavi, San Diego, CA (US); Chenyu Zhang, Rosemead, CA (US); Kyle Atlas, Huntington Beach, CA (US)

(72) Inventors: Morteza Naghavi, San Diego, CA (US); Chenyu Zhang, Rosemead, CA (US); Kyle Atlas, Huntington Beach, CA (US)

(21) Appl. No.: 19/390,544

(22) Filed: Nov. 15, 2025

Related U.S. Application Data
(60) Provisional application No. 63/721,486, filed on Nov. 16, 2024.

(51) Int. Cl. G16H 30/40; G06V 10/30; G06V 10/70

(52) U.S. Cl. CPC G16H 30/40; G06V 10/30; G06V 10/70

(57) ABSTRACT

Method and system for scanner-normalized quantitative CT analysis through RDU calibration to analyze plaque-level characterization and longitudinal stability tracking for early detection of sub-clinical disease and monitoring of therapy response, and to reuse of existing CT scans for multi-disease prevention.

### CROSS-REFERENCE TO RELATED APPLICATIONS

This application claims priority to U.S. Provisional Patent Application No. 63/721,486, filed Nov. 16, 2024, entitled “AI-Enabled Early Detection and Prevention of Adverse Health Outcomes,” which is incorporated by reference in its entirety.

### FIELD OF INVENTION

The present invention relates to medical image analysis and artificial intelligence (AI) systems for cardiometabolic risk assessment. More specifically, it pertains to AI-enabled methods, systems, and computer program products for quantitative analysis of non-contrast computed tomography (CT) images—including coronary artery calcium (CAC) scans and low-dose chest CT scans—to automatically detect and characterize coronary calcification, assess plaque stability over time, and opportunistically extract extracoronary and extracardiac biomarkers for early detection and prevention of cardiometabolic disorders. The system integrates multiple imaging biomarkers—including coronary calcium burden (AI-CAC), plaque stability, hepatic and muscular fat indices, emphysema, aortic and valvular calcification, pericardial fat, and bone density—to generate quantitative features. These features are analyzed by event-specific AI-CVD models ((AI-CVD-Heart Failure (HF), AI-CVD-Atrial Fibrillation (AF), AI-CVD-Stroke, AI-CVD Coronary Heart Disease (CHD), AI-CVD-All-CVD, and AI-CVD-Mortality)) employing survival-learning architectures to predict individualized risks for HF, AF, stroke, cardiovascular disease, and mortality.

The present invention discloses methods that can be implemented in hospital PACS, radiology workstations, or cloud-based screening networks to enable population-scale cardiovascular and metabolic risk assessment.

### BACKGROUND

Coronary artery calcification, quantified by the Agatston scoring method, has become a standard tool for risk stratification of atherosclerotic cardiovascular disease (ASCVD). The “power of zero”—absence of CAC indicating very low near-term risk—has been validated across multiple cohorts. However, it lack sufficient sensitivity to detect “soft plaques” which are seen as the culprit for coronary events in younger than 50-60 year old patients. For example, in the Multi-Ethnic Study of Atherosclerosis (MESA) over a 15-year follow up 80 people out of 3260 participants with Agatston score of zero at baseline developed an adverse coronary event. This number at 5-year follow up which is recommended interval for repeating a coronary calcium scan was 26. This translates to about 10% of all coronary events in MESA which is a substantial number and leaves much to be desired. This lack of sufficient sensitivity is because conventional Agatston scoring suffers from several critical limitations as it uses a fixed intensity threshold (130 HU) and slice-based area summation, making it sensitive to image noise, reconstruction kernel, and scanner variability. It further provides no per-plaque detail (location, density distribution, or vessel association), preventing analysis of heterogeneous plaque stability, and often increases under statin therapy, even when plaques stabilize or regress, creating clinical confusion about progression versus healing.

Meanwhile, tens of millions of non-contrast chest CT scans—performed for lung cancer screening or other indications—remain an untapped resource for opportunistic cardiovascular screening. These scans contain valuable information about the heart, liver, and skeletal muscle that correlates with disease outcomes but is rarely analyzed.

Existing Artificial Intelligence/Machine Learning (AI/ML)-based calcium scoring methods largely replicate Agatston calculations and fail to incorporate Cross-scanner calibration using in-scan references to create scanner-independent quantitative maps, Plaque-level tracking across serial scans to distinguish pharmacologic stabilization (density increase with constant area) from true progression (area increase), Integration of extracardiac biomarkers, such as liver attenuation (hepatic steatosis) and muscle attenuation (myosteatosis), which are known predictors of metabolic and cardiac disease.

Therefore, there is an unmet need for an AI-based system that overcomes the constraints of the Agatston method, provides plaque-level intelligence, enables longitudinal tracking, and extends risk prediction beyond coronary atherosclerosis.

Furthermore, Recent advances in machine learning and image-based phenotyping have revealed that cardiovascular disease risk is influenced by a broad spectrum of imaging biomarkers extending well beyond coronary calcification. Non-contrast and contrast-enhanced CT scans of the chest contain quantitative information about the heart, lungs, vessels, liver, muscle, fat, and bone that reflect underlying metabolic and vascular health. Parameters such as emphysema burden, aortic and pulmonary dimensions, valvular and aortic calcification, pericardial and visceral fat, hepatic steatosis, skeletal-muscle attenuation, and thoracic bone mineral density have each been shown to correlate with adverse outcomes including heart failure, atrial fibrillation, stroke, and all-cause mortality. However, no existing system integrates these features into a unified predictive framework capable of producing individualized, multi-event risk estimates. Accordingly, there remains a need for an artificial-intelligence platform that simultaneously analyzes coronary, pulmonary, vascular, metabolic, and skeletal biomarkers from a single CT study to generate comprehensive, clinically actionable risk profiles.

### SUMMARY OF THE INVENTION

An AI pipeline performs coronary segmentation on non-contrast CT, calibrates intensities to relative density units (RDU) via in-scan references, applies noise-adaptive filtering, detects plaques, computes plaque-level features, and outputs Agatston 2.0 (AI-CAC) and a Plaque Stability Index (PSI) from longitudinal area-density trajectories. The same CT yields quantitative imaging biomarkers for osteoporosis, myosteatosis, hepatosteatosis, emphysema, and other measures to inform multi-disease risk models, exported via DICOM SR/FHIR. Furthermore, the AI can reliably predict obstructive coronary artery disease (stenosis>50%), ischemia, non-calcified plaques, and plaques with high risk (vulnerable plaque) features. Agatston 2.0 (AI-CAC) significantly increases the “Power of Zero”. For example, in MESA it up-classified 68% of Agatston CAC zero patients who experienced a coronary event into AI-CAC greater than zero.

In one embodiment, computer-implemented methods and systems collectively referred to as Agatston 2.0/AI-CAC perform automatic segmentation of the coronary artery tree on non-contrast CT, intensity calibration using in-scan references to create Relative Density Units (RDU) independent of scanner parameters, noise-adaptive spatial filtering to enhance plaque detectability, plaque-level detection and labeling with volumetric and density statistics, derivation of a Plaque Stability Index (PSI) that quantifies density-area co-trajectories over serial scans to distinguish pharmacologic stabilization from progression, and concurrent extraction of Liver Attenuation Index (LAI) and Myosteatosis Index (MI) for metabolic profilin as well other HU density-based indices such as lung hypodensity (emphysema) and hypodensity (ILA) index.

In some embodiments, imaging-derived biomarkers are input to a predictive engine comprising multiple event-specific models collectively referred to as AI-CVD-HF, AI-CVD-AF, AI-CVD-Stroke, AI-CVD-All-CVD, and AI-CVD-Mortality. These models employ supervised and survival-learning architectures—including random survival forests, Cox proportional-hazards regression, gradient-boosted survival trees, and deep neural survival networks—to generate individualized risk predictions for heart failure, atrial fibrillation, stroke, cardiovascular disease, and all-cause mortality.

In certain embodiments, the AI-CVD framework further integrates outputs from the event-specific models through a Fusion Risk Engine, which produces a composite cardiopulmonary-metabolic risk score and generates an interactive, structured report. The system supports continuous and federated learning across distributed datasets, allowing model refinement without direct transfer of patient data, thereby maintaining privacy while enhancing generalization.

In certain embodiments, the system performs federated or continuous learning by retraining the AI-CVD models on distributed imaging databases without centralized transfer of patient data, thereby maintaining data privacy while improving model generalization. The model outputs are stored in a structured format and used to trigger follow-up actions such as cardiac referral, therapy intensification, or additional imaging. These models extend the utility of chest CT scans from static structural assessment to dynamic risk prediction for multiple cardiovascular and metabolic outcomes, providing a foundation for precision prevention and longitudinal monitoring.

In some embodiments, the AI-CVD platform is implemented as a cloud-based or on-premises software system accessible through standard clinical workflows, enabling opportunistic risk assessment from routine CT scans and providing actionable recommendations for prevention, follow-up imaging, or therapy optimization.

### BRIEF DESCRIPTION OF DRAWINGS

The embodiments herein will be better understood from the following detailed description with reference to the drawings, in which:

FIG. 1 is a flowchart that illustrates input non-contrast CT→preprocessing (cropping, resampling, normalization)→coronary artery segmentation (AI-based)→intensity calibration (phantom/fat reference)→noise filtering→plaque detection→per-plaque feature extraction→AI-CAC computation→report generation.

FIG. 2 is a 3D rendering of segmented coronary arteries (LM, LAD, LCx, RCA) from a non-contrast CT with labeled plaques, each assigned a unique ID and color-coded by density or volume.

FIG. 3 is a graph showing plaque-level area and density trajectories across baseline and follow-up scans; shaded regions illustrate statin stabilization (density↑, area stable/↓) vs. progression (area↑).

FIG. 4 illustrates a whole-liver segmentation from chest CT with histogram of voxel intensities within [−30,70] HU; LAI represented as percentage <40 HU.

FIG. 5. shows liver Attenuation Index (LAI) distribution with whole-liver segmentation from a cardiac scan showing examples of high and low LAI on both ends.

FIG. 6 shows a liver Attenuation Index (LAI) in patients with and without diabetes measured using the same coronary artery calcium scans used for coronary artery calcium score.

FIG. 7. illustrates Kaplan-Meier cumulative-incidence curves showing the additive value of high Liver Attenuation Index (LAI) in patients with high coronary artery calcium score.

FIG. 8. is Thoracic Myosteatosis Measurement showing Axial slice showing segmented paraspinal and pectoral muscles with overlay of HU color map; bar graph showing Myosteatosis quartiles versus AF/HF incidence plus two examples of high and low Myosteaotosis.

FIG. 9. compares Myosteatosis and Hepatosteatosis for Predicting New Onset Diabetes Melitus (DM) that Non-contrast CAC scan based Myosteatosis and Hepatosteatosis indices were used to predict new onset diabetes in non-glycemic non-obese patients. Hepatosteatosis measured by Liver Attenuation Index (LAI) showed a much stronger predictive value than Myosteatosis.

FIG. 10. shows a simplified Integrated Cardiometabolic Risk Dashboard that composite report output is combined with AI-CAC (Agatston 2.0) score, PSI classification, LAI, MI, and predicted risk for CHD, HF, AF, DM, and mortality.

FIG. 11. is a template for comprehensive AI-CVD Quantitative Imaging and Risk Dashboard where a composite report shows all components of AI-CVD quantitative measurement analysis and predicted risk for All CVD, CHD, HF, AF, LVH, DM, and mortality.

FIG. 12. is Coronary Analysis Quantitative Imaging UI showing interactive user interface for AI-CAC based plaque analysis in non-contrast and contrast-enhanced CT scans and monitoring changes over time.

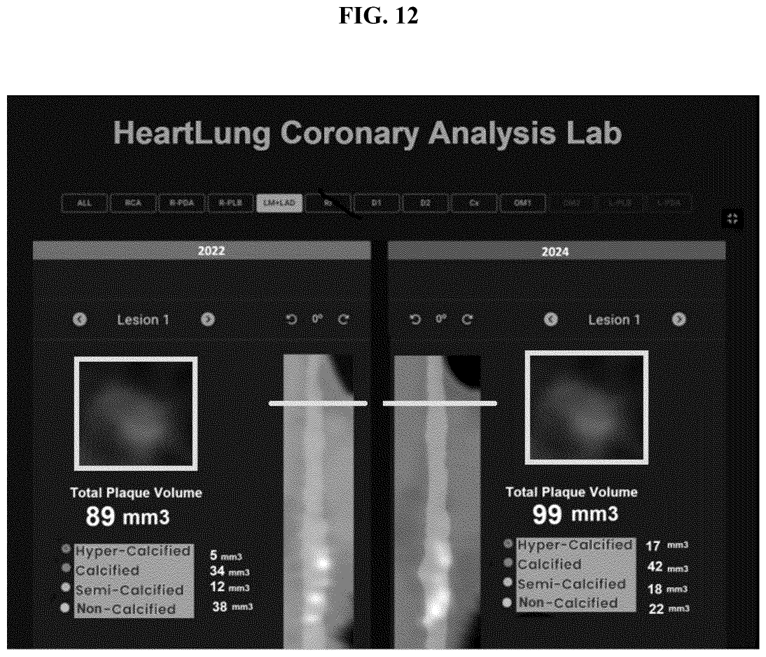

FIG. 13. is a schematic of neural network integrating AI-CAC, LAI, MI, and adiposity inputs for multi-disease risk prediction.

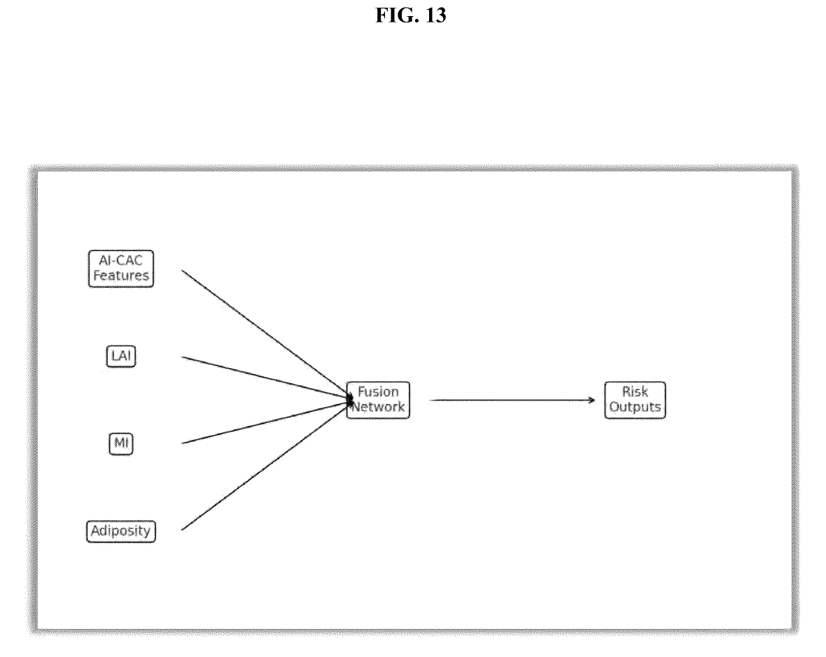

FIG. 14. Kaplan-Meier Cumulative-Incidence Curves for AI-CVD components-cumulative incidence of CVD events among the top quartile (high risk) group of each AI-CVD measurement over 18-years in the Multi-Ethnic Study of Atherosclerosis (MESA)

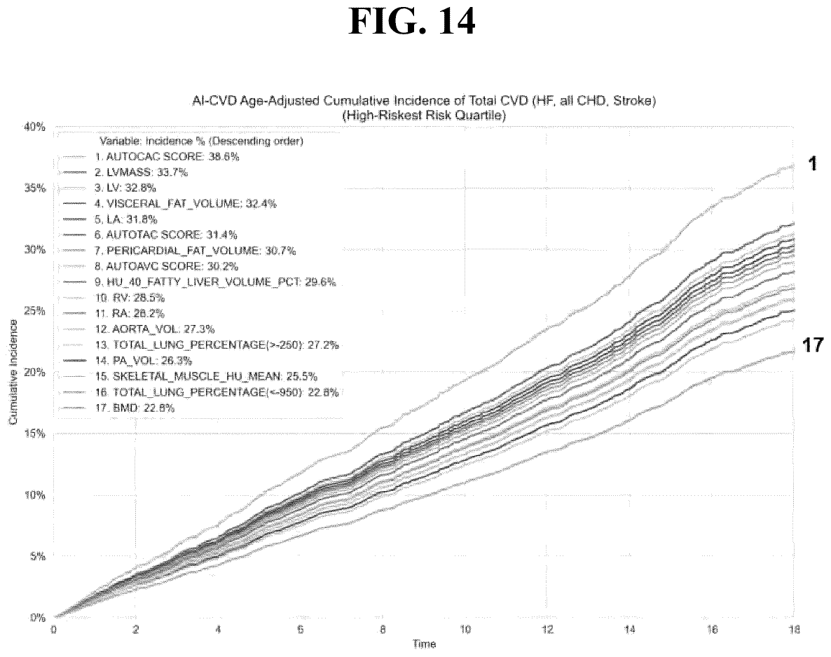

FIG. 15. Multi-Factorial Analysis of Components of AI-CVD by Contributing Factors-AI-CVD variables listed by importance for 10-year outcomes including heart failure, atrial fibrillation, all CVD and mortality based on Multi-Ethnic Study of Atherosclerosis (MESA)

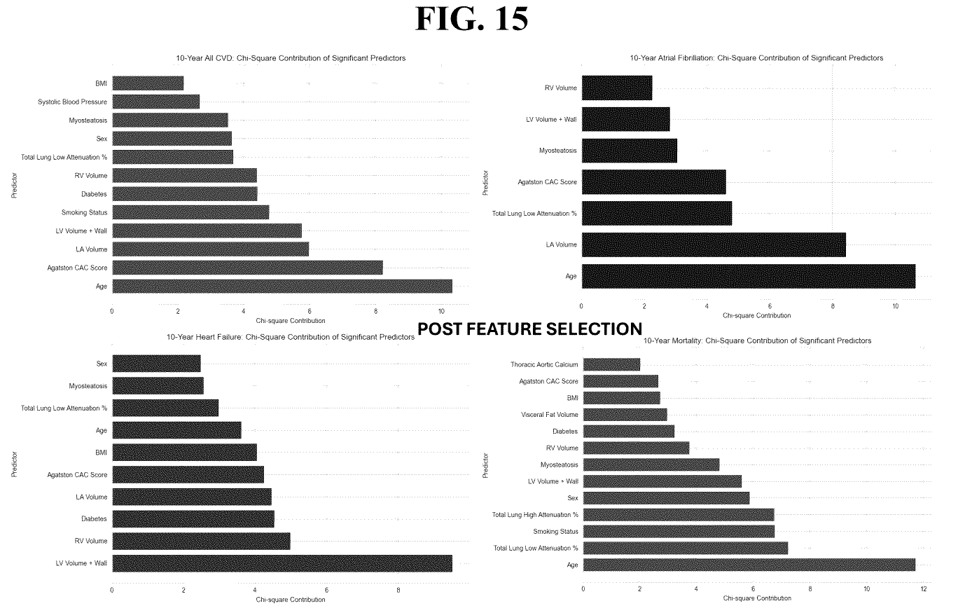

FIG. 16. Key Advantages of AI-CAC over Traditional Agatston Score shows the added value of AI-CAC by increasing the “Power of Zero” in Agatston CAC zero population and enabling monitoring changes in plaque stability by monitoring changes in the density of coronary arteries and previously identified plaques.

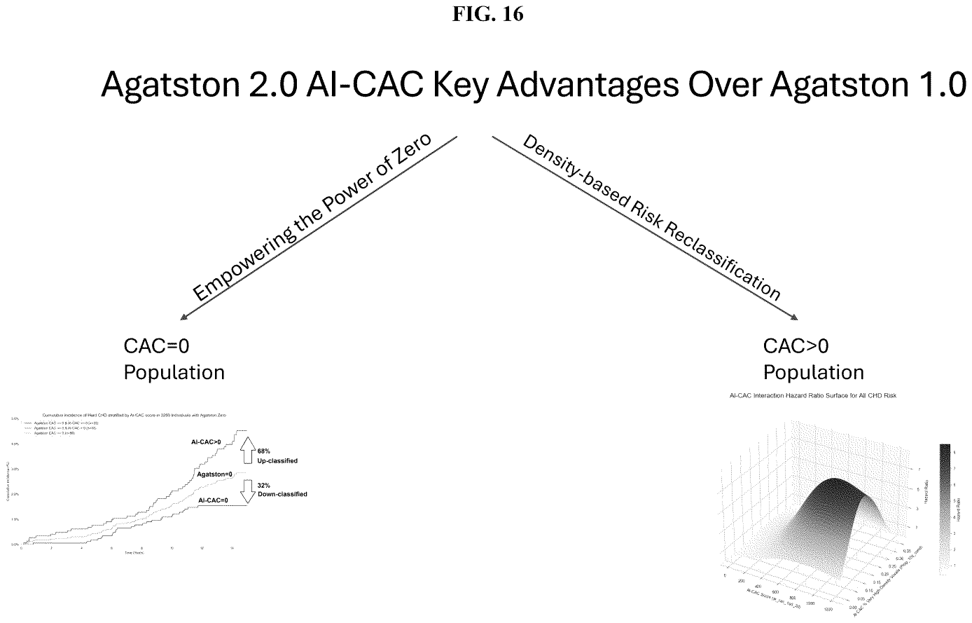

FIG. 17. Kaplan-Meier cumulative-incidence curves showing 15-year CHD event rates among participants with Agatston=0, stratified by AI-CAC=0 vs AI-CAC>0, demonstrating enhanced negative-predictive power (“Power of Zero”) based on Multi-Ethnic Study of Atherosclerosis (MESA)

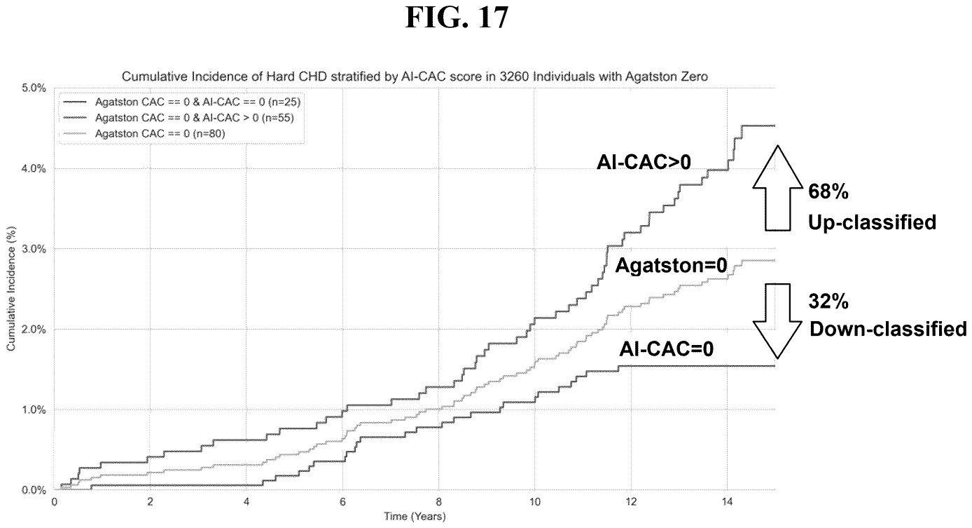

FIG. 18. Table comparing the “Power of Zero” between Agatston CAC zero and AI-CAC zero over 5, 10- and 15-year CHD event rates among participants, demonstrating enhanced negative-predictive power (“Power of Zero”) based on participants in the Multi-Ethnic Study of Atherosclerosis (MESA)

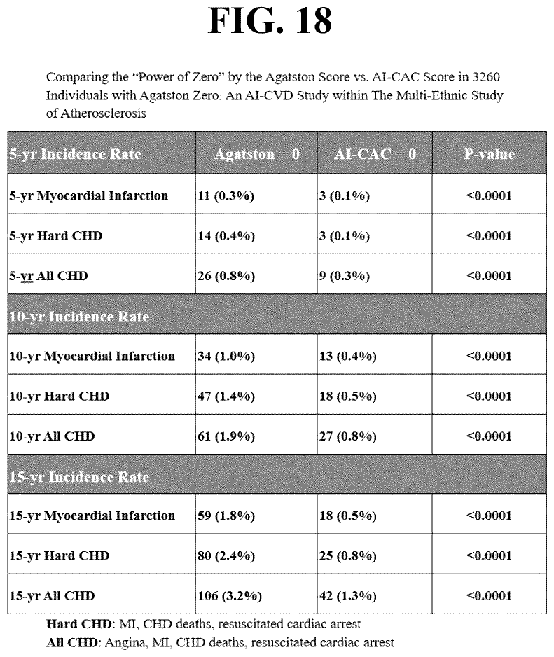

FIG. 19. Kaplan-Meier cumulative-incidence curves showing 15-year CHD event rates among participants with Agatston=0, stratified by AI-CAC=0 vs AI-CAC>0, demonstrating enhanced negative-predictive power (“Power of Zero”) based on combined Multi-Ethnic Study of Atherosclerosis (MESA) and Framingham Heart Study (FHS)

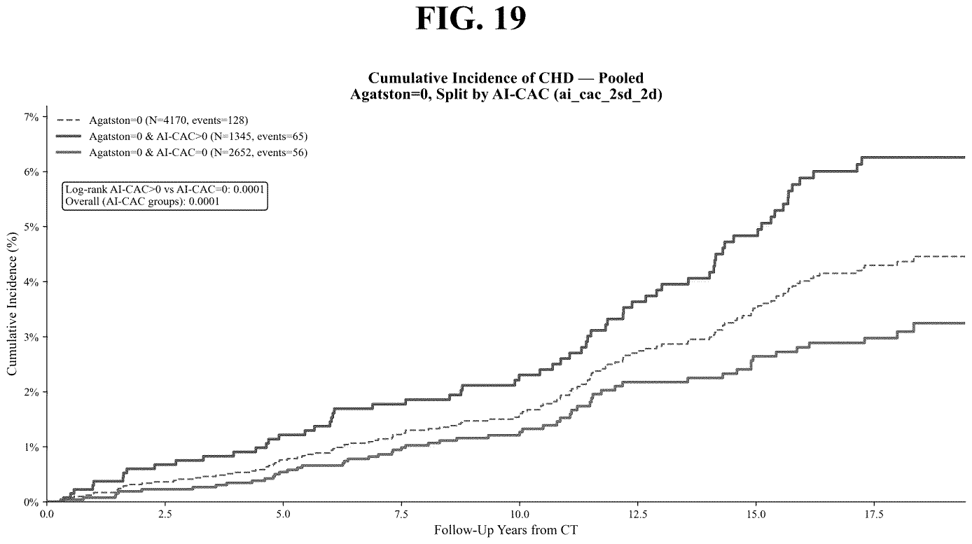

FIG. 20. Age-Adjusted Kaplan-Meier Cumulative-Incidence Curves demonstrating lower event rates in higher plaque density among participants of the Multi-Ethnic Study of Atherosclerosis (MESA) with AI-CAC>400

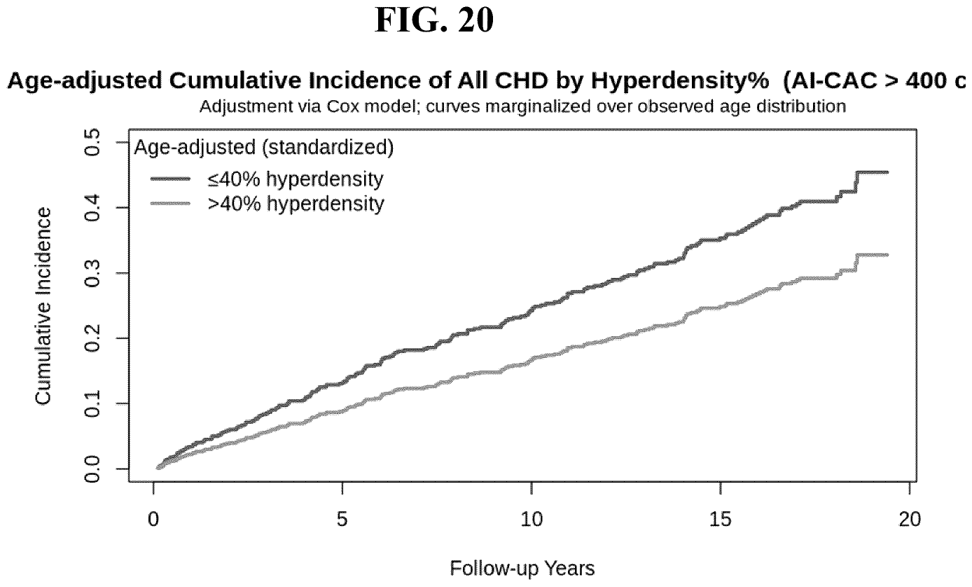

FIG. 21. Scatterplot and regression of plaque hyperdensity index versus CHD risk, illustrating that higher mean plaque density corresponds to lower event rates.

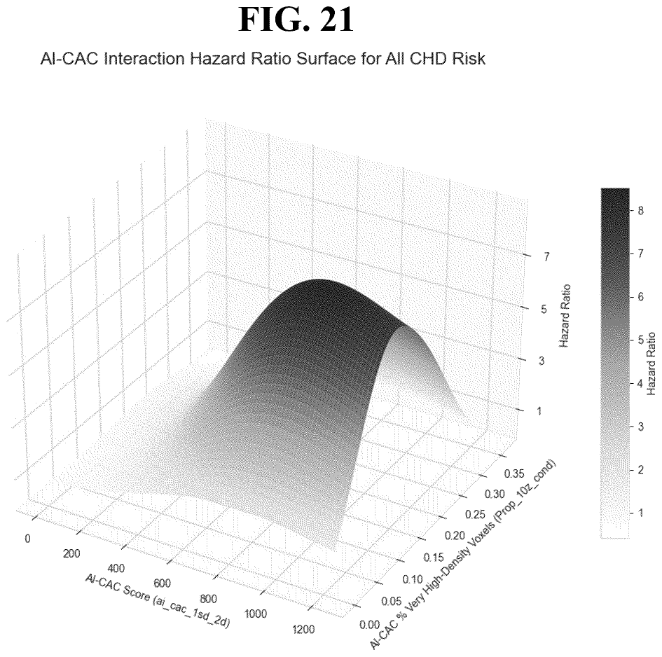

FIG. 22. Three-Dimensional Histogram showing the relationship between plaque hyperdensity index versus AI-CAC and associated CHD risk.

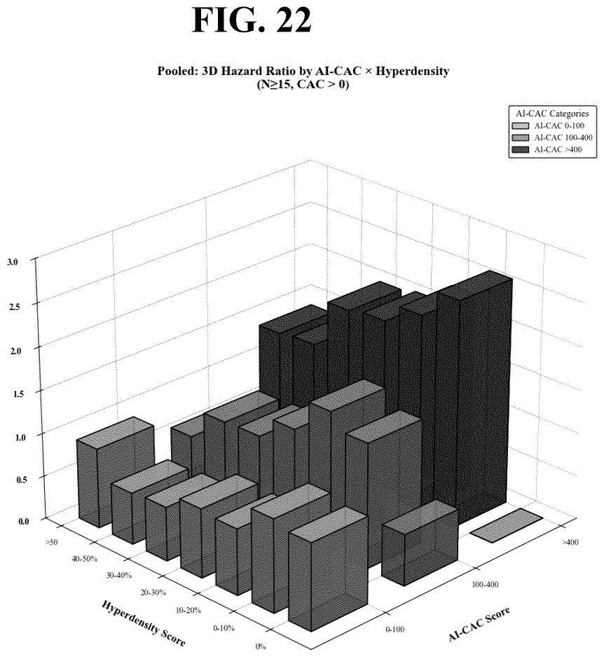

FIG. 23. Plaque Volume-Adjusted Hyperdensity Index in statin naïve patients versus statin users demonstrating the effect of statin on reducing risk while increasing plaque density.

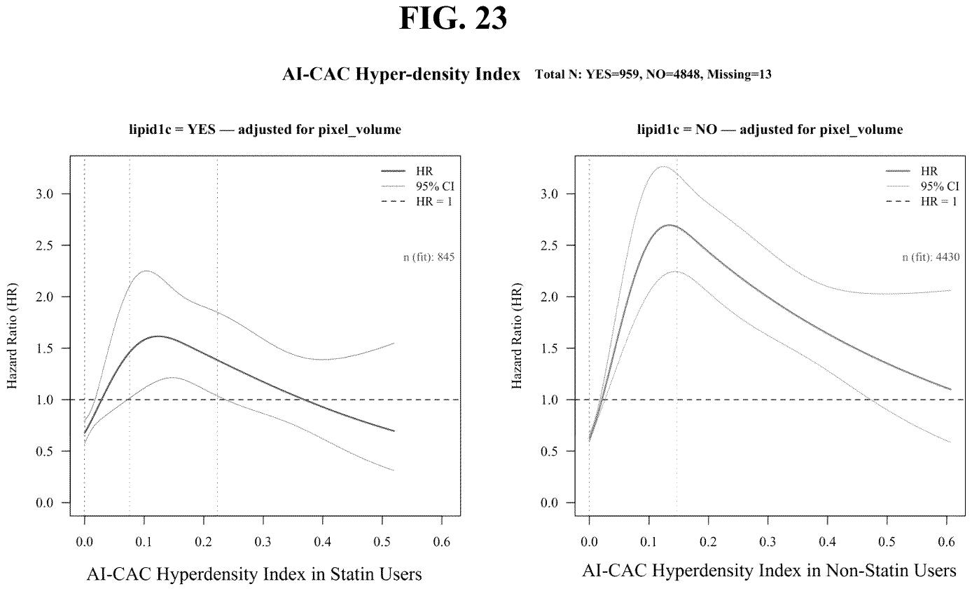

FIG. 24. Phantom-based vs. Subcutaneous Fat-based Calibration the correlation plots show a strong agreement between AI-CAC calculated based on an external calibration phantom versus an internal calibration medium.

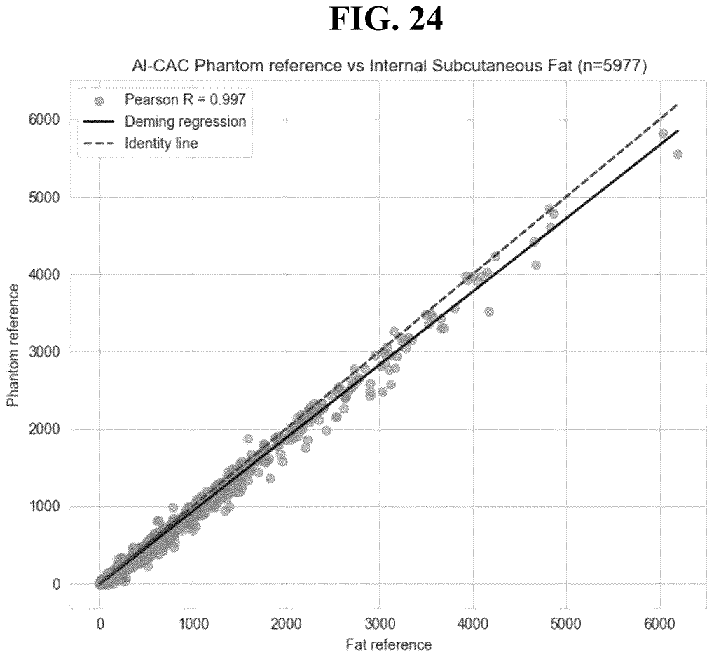

FIG. 25. AI-CAC Coronary Segmentation vs. Contrast-enhanced Coronary CT Angiography (CCTA) the image on the left shows AI-CAC based coronary segmentation in s non-contrast CAC scan and the image on the right shows existing coronary segmentation obtained from a contrast-enhanced coronary CT angiography (CCTA) in the same patient demonstrating substantially similar characterization of left main (LM), left anterior descending (LAD), left circumflex (LCX) and right coronary artery (RCA).

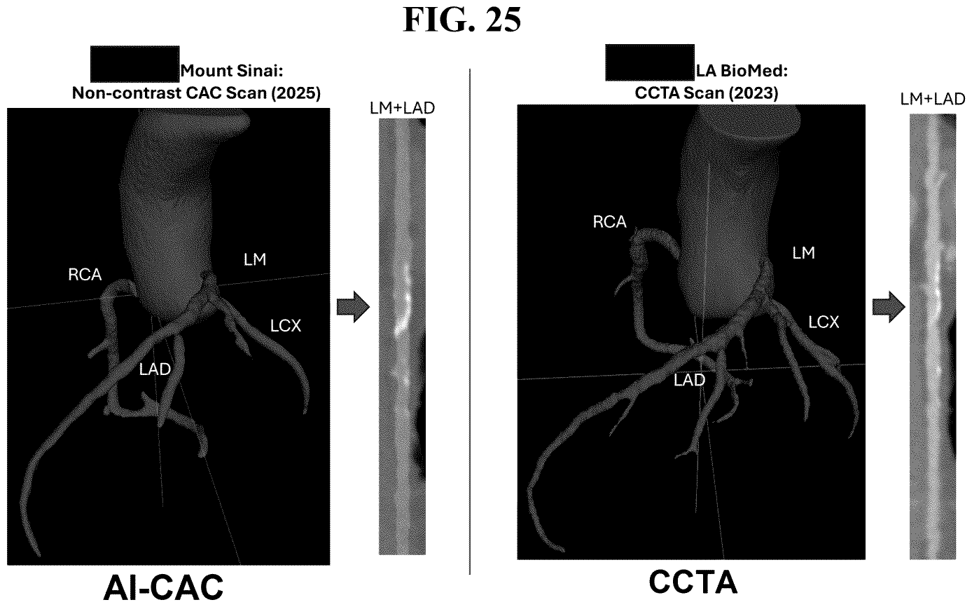

FIG. 26. Receiver-operating-characteristic (ROC) and precision-recall curves comparing AI-CVD-HF versus PREVENT-HF models, showing superior AUC (0.84 vs 0.79) and AUPRC (+44%) for heart-failure prediction.

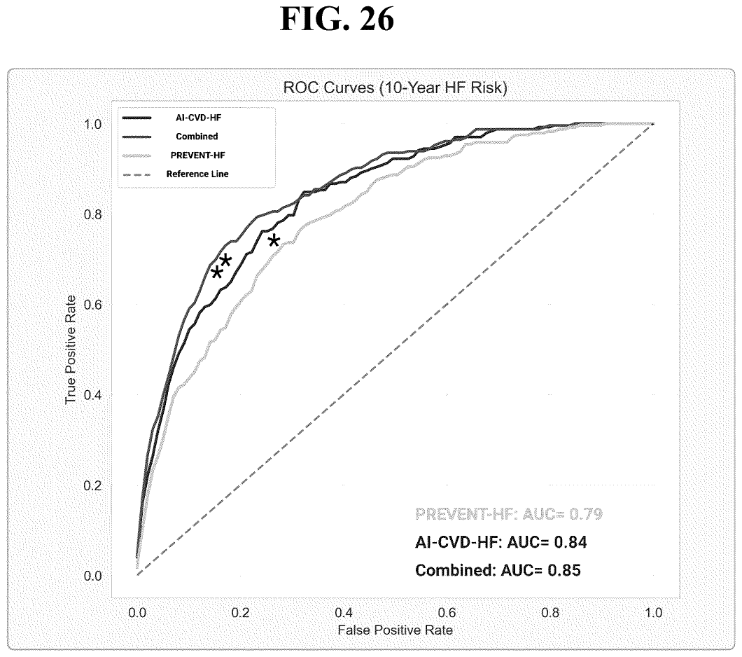

FIG. 27. ROC and calibration plots comparing AI-CVD-AF versus CHARGE-AF, showing improved 5-year AUC (0.78 vs 0.75) and 45% higher AUPRC.

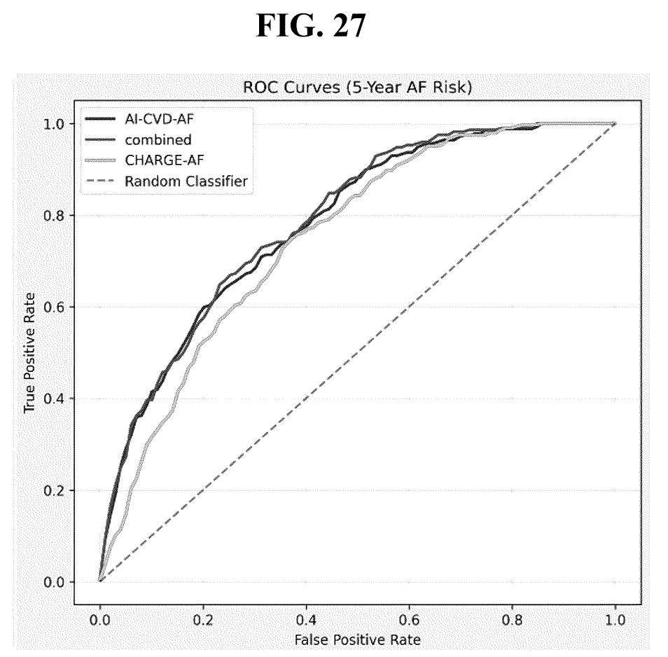

FIG. 28. Left-To-Right Ratio in Cardiac Chamber Volumetry and Heart Failure shows strong predictive power of LV/RV, LA/RA, and LA/RV ratio for predicting heart failure.

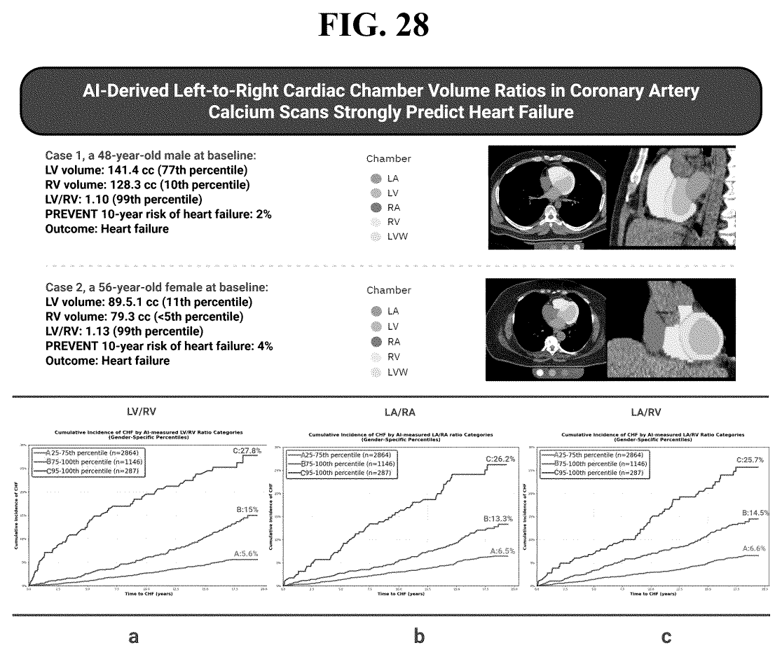

FIG. 29. Thoracic Myosteatosis and Chronic Obstructive Pulmonary Disease (COPD) shows strong predictive power of Myosteatosis in thoracic skeletal muscle for predicting clinically diagnosed COPD.

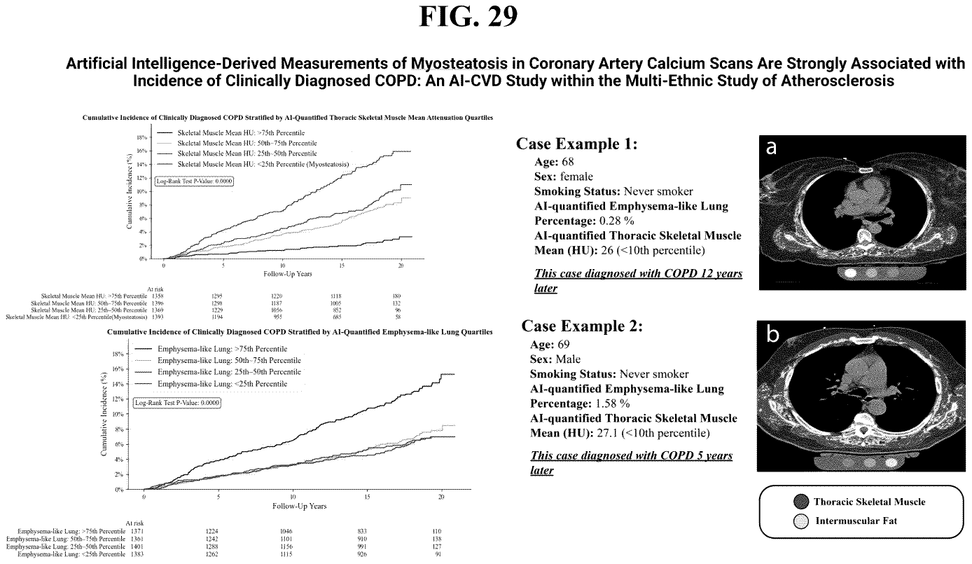

FIG. 30. AI-CVD Risk Score comprising various measurements within the AI-CVD platform including AI-CAC, cardiac chambers volume, thoracic aortic valve and wall calcification, pericardial fat, lung density, liver density, and bone mineral density, and traditional cardiovascular risk factors.

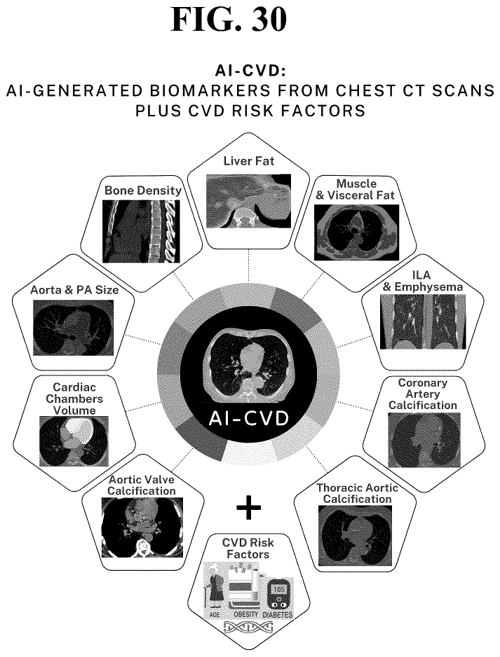

FIG. 31. AI-CVD CHD Risk Score comprising various measurements within the AI-CVD platform that predicts obstructive coronary artery disease (stenosis>50%), ischemia, non-calcified plaques and high-risk (vulnerable) plaque features.

FIG. 32. AI-CVD Lung Low Attenuation Index flags cases with emphysema like disorders.

FIG. 33. AI-CVD Lung High Attenuation Index flags cases with interstitial lung disease and other disorders of lung parenchyma that result in increasing lung density.

FIG. 34. AI-CVD Coronary Artery Calcification flags cases with atherosclerotic coronary artery disease and report both Agatston score and AI-CAC (Agatston 2.0) score.

FIG. 35. AI-CVD Aortic Wall and Valve Calcification flags cases with atherosclerotic in aorta as well as calcified valves that result in aortic stenosis.

FIG. 36. AI-CVD Aorta and Pulmonary Sizing flags cases with enlarged aorta (aortic aneurysm) and enlarged pulmonary trunk due to pulmonary hypertension.

FIG. 37. AI-CVD Aortic Profiling flags cases with aortic aneurysm through thoracic and abdominal cavities both in non-contrast and contrast-enhanced CT scans.

FIG. 38. AI-CVD Liver Attenuation Index (LAI) flags cases with fatty liver disease.

FIG. 39. AI-CVD Muscle and Fat Composition flags cases with high visceral fat, sarcopenia and myosteatosis.

FIG. 40. AI-CVD Pericardial Fat flags cases with high pericardial fat who are at risk of cardiovascular disease.

FIG. 41. AI-CVD Bone Mineral Density flags cases with osteopenia and osteoporosis at risk of fracture.

FIG. 42—Multi-disease risk integration map schema illustrating combined output of the AI-CVD system generating a composite cardiopulmonary-metabolic score.

### DETAILED DESCRIPTIONS

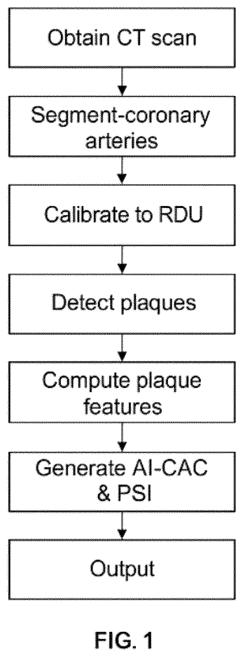

Referring to FIG. 1, a non-contrast CT dataset (100) is provided as input. The dataset passes through sequential modules: preprocessing (110), coronary segmentation (120), intensity calibration (130), noise-adaptive filtering (140), plaque detection (150), feature extraction (160), stability and biomarker computation (170), and report generation (180).

Still referring to FIG. 1, a non-contrast CT dataset (100) is provided as input. The dataset passes through sequential modules: preprocessing (110), coronary segmentation (120), intensity calibration (130), noise-adaptive filtering (140), plaque detection (150), feature extraction (160), stability and biomarker computation (170), and report generation (180).

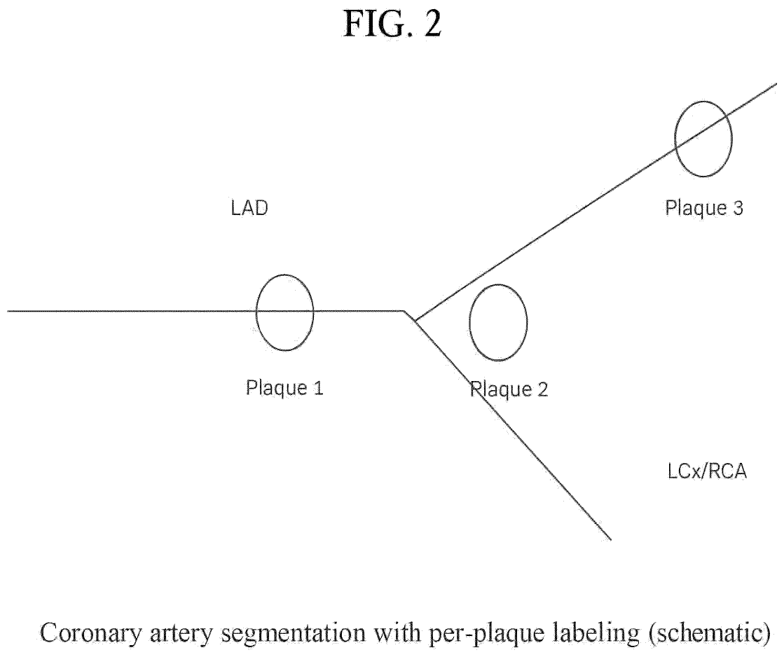

As illustrated in FIG. 2, a trained three-dimensional neural network (200)—for example a 3D U-Net or transformer-based model—produces a coronary-artery probability map. Training employs paired datasets of non-contrast and contrast-enhanced CCTA volumes with transferred vessel labels. Post-processing includes connected-component filtering and centerline extraction to identify left main (LM), left anterior descending (LAD), left circumflex (LCX), and right coronary artery (RCA) segments

### Preprocessing

Input volumes are normalized to Hounsfield range (−1000 to 1500 HU) and resampled to isotropic voxels. A body-part locator isolates the thoracic region. Orientation is standardized (LPS coordinate system) to ensure consistent downstream alignment.

### Coronary Segmentation

As illustrated in FIG. 2, a trained three-dimensional neural network—for example a 3D U-Net or transformer-based model—produces a coronary-artery probability map. Training employs paired datasets of non-contrast and contrast-enhanced CCTA volumes with transferred vessel labels. Post-processing includes connected-component filtering and centerline extraction to identify left main (LM), left anterior descending (LAD), left circumflex (LCx), and right coronary artery (RCA) segments.

### Intensity Calibration to Relative Density Units (RDU)

A calibration module (300) detects a subcutaneous-fat reference or a 100 HU external calibration phantom if available. Using measured mean and variance, it constructs a monotonic transformation T(HU)→RDU that compensates for scanner-specific offsets and noise. RDUs are dimensionless and comparable across CT scanners and protocols.

### Noise-Adaptive Filtering

Before plaque detection, a 3D anisotropic filter suppresses isolated noisy voxels while preserving edge continuity. Filter parameters are derived from estimated image noise σ² measured in fat regions. The result is a smooth but edge-sharp coronary volume.

### Plaque Detection and Feature Extraction

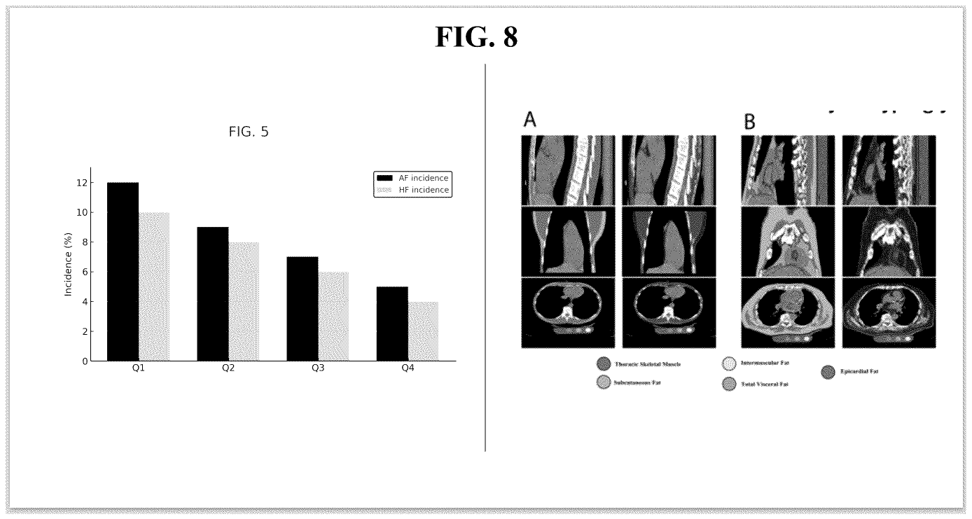

Still shown in FIG. 2, within the segmented coronary mask, voxels exceeding a context-adaptive RDU threshold are grouped into connected components representing plaques. Each plaque is characterized by 3D volume (V), surface area (A), centroid (x,y,z); mean, median, minimum, maximum, and percentile RDUs; density heterogeneity (H=σ/μ); and vessel-segment identity. Each plaque is assigned a persistent identifier (IDp) for cross-temporal correspondence (see FIG. 8).

### AI-CAC Score Computation

A plaque's contribution to total burden is calculated as

$${S}_{p} = {\int }_{{V}_{p}} f ( RDU ) dv$$

where f is a nonlinear weighting emphasizing higher densities. The overall AI-CAC score is the sum ΣSₚ across all plaques. This replaces the fixed 130 HU threshold with a calibrated, continuous model.

### Plaque Stability Index (PSI)

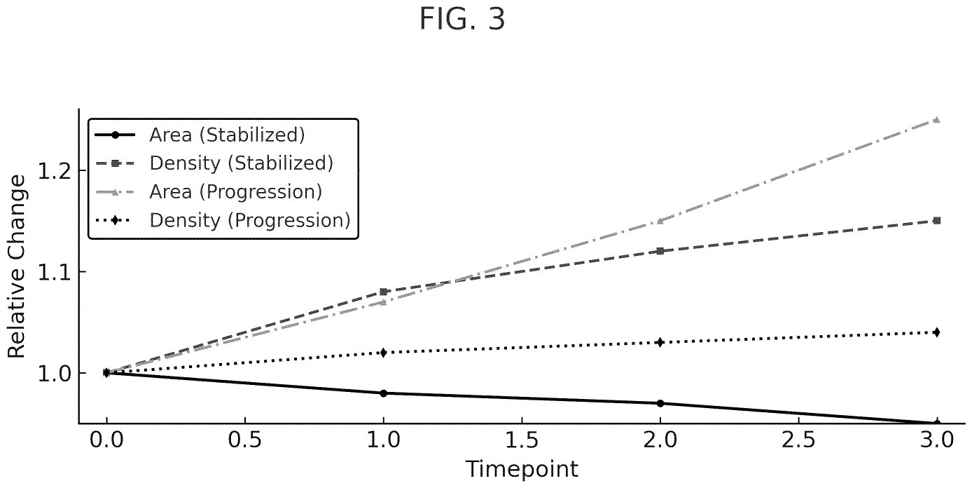

For longitudinal studies (FIG. 3), plaque p has baseline (B) and follow-up (F) features (AB, AF, DB, DF) representing area (A) and mean density (D). A Plaque Stability Index is defined as

$${PSI}_{p} = {w}_{1} \frac{Δ D}{{D}_{B}} - {w}_{2} \frac{Δ A}{{A}_{B}}$$

where ΔD=(DF−DB) and ΔA=(AF−AB); weights w₁, w₂ are empirically tuned. Positive PSI values indicate stabilization (density ↑ with area ↔ or ↓); negative values indicate progression (area ↑).

Alternative embodiments use a recurrent neural network trained on plaque-trajectory pairs to predict categorical stability.

### Liver Attenuation Index (LAI)

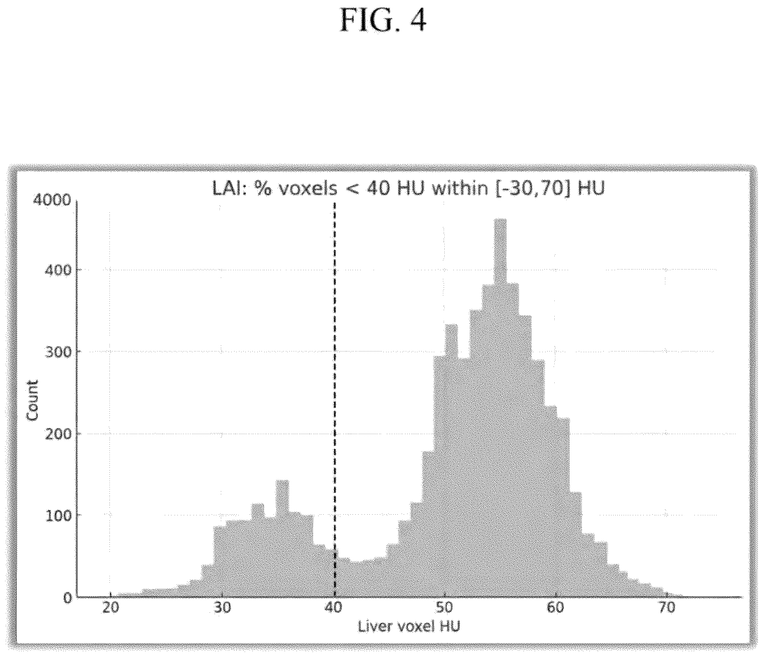

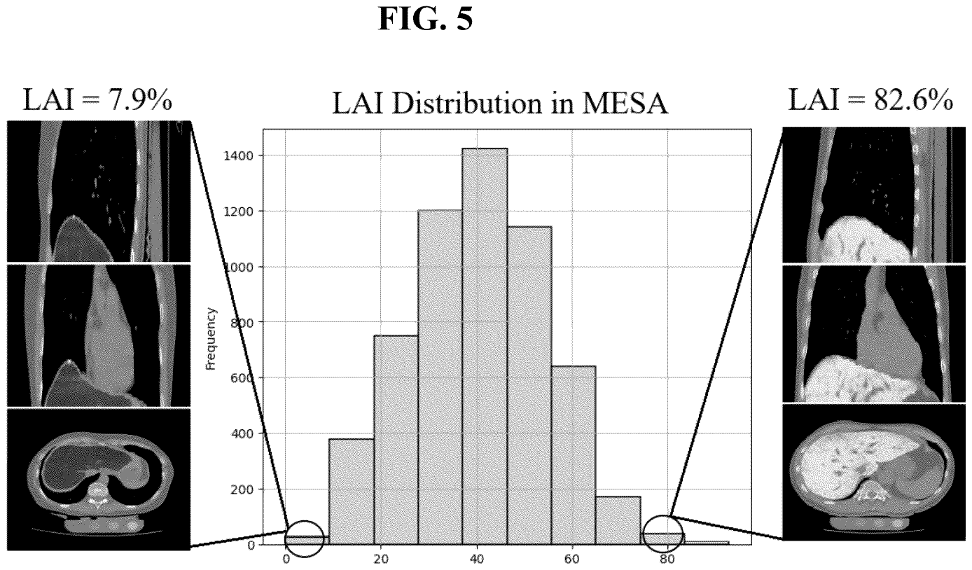

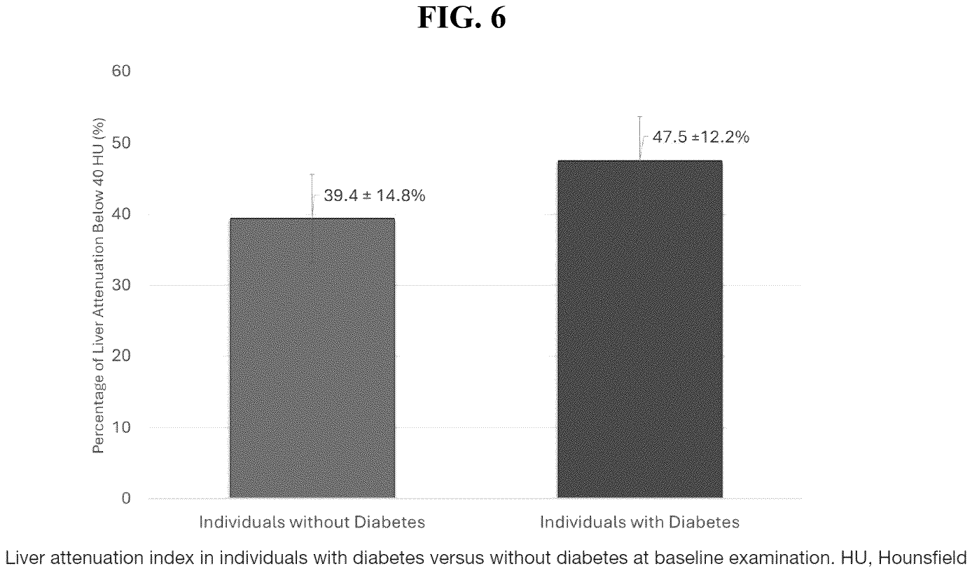

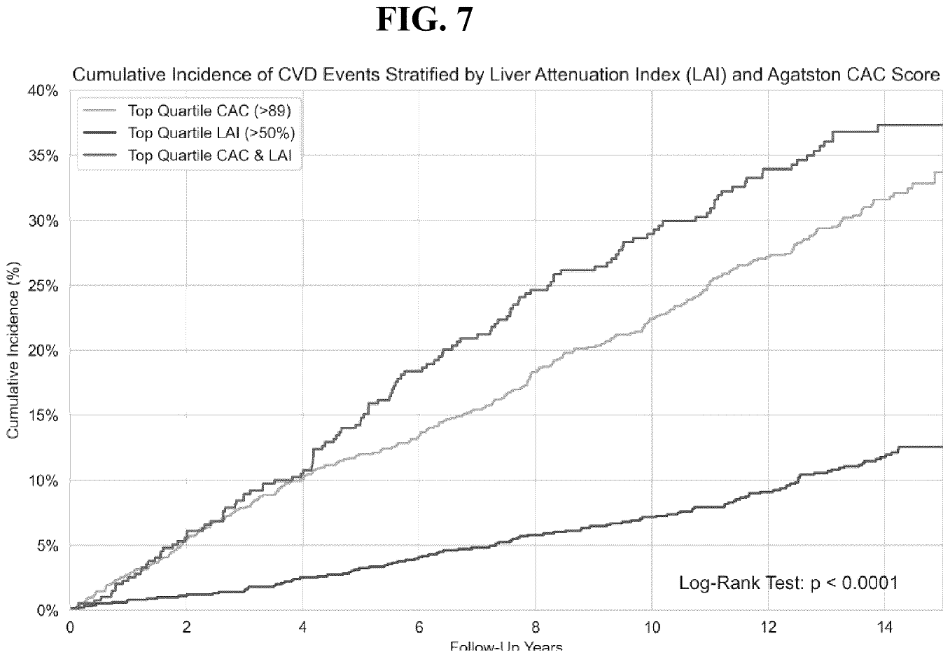

As shown in FIG. 4-7, the liver is segmented automatically. Voxels within [−30, 70] HU are analyzed to compute the proportion below 40 HU. The LAI=(% of voxels<40 HU) quantifies hepatic steatosis; lower attenuation corresponds to higher fat infiltration with examples of low LAI=7.9% to high LAI-82.6% plus a histogram showing significantly higher LAI in diabetes patients versus non-diabetes. Additionally FIG. 7 shows higher risk of CVD events when both CAC score and LAI are high.

### Myosteatosis Index (MI)

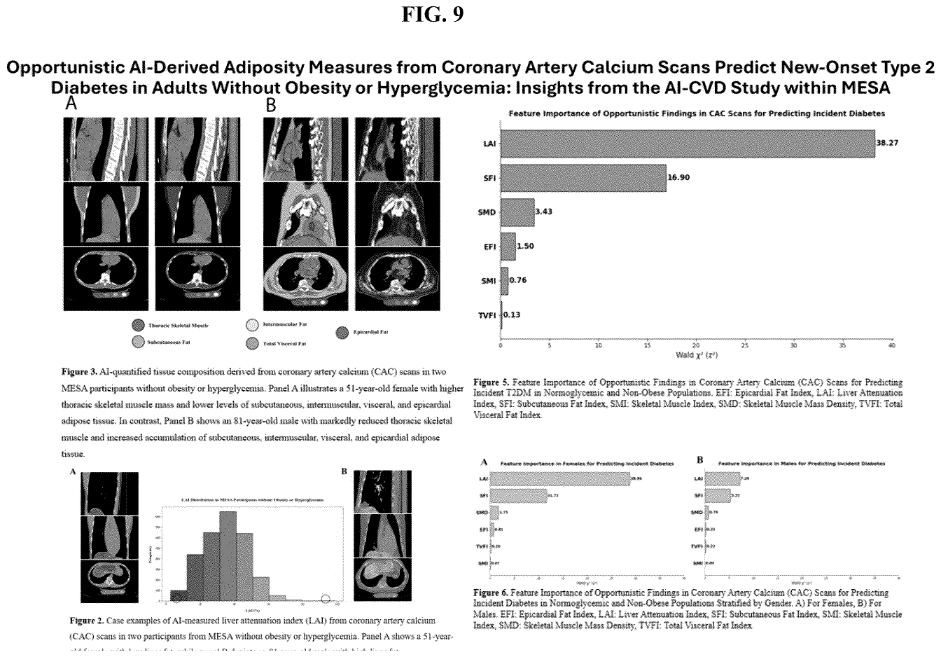

Referring to FIG. 8,9, thoracic skeletal muscle—paraspinal and pectoral regions—is segmented. Mean muscle HU or inferred fat fraction defines a Myosteatosis Index. Lower MI values indicate fatty infiltration and correlate with frailty, heart failure, and arrhythmic risk. Furthermore FIG. 9 illustrates the relative contribution of MI versus LAI and subcutaneous fait index (SFI).

### Integration and Multi-Disease Prediction

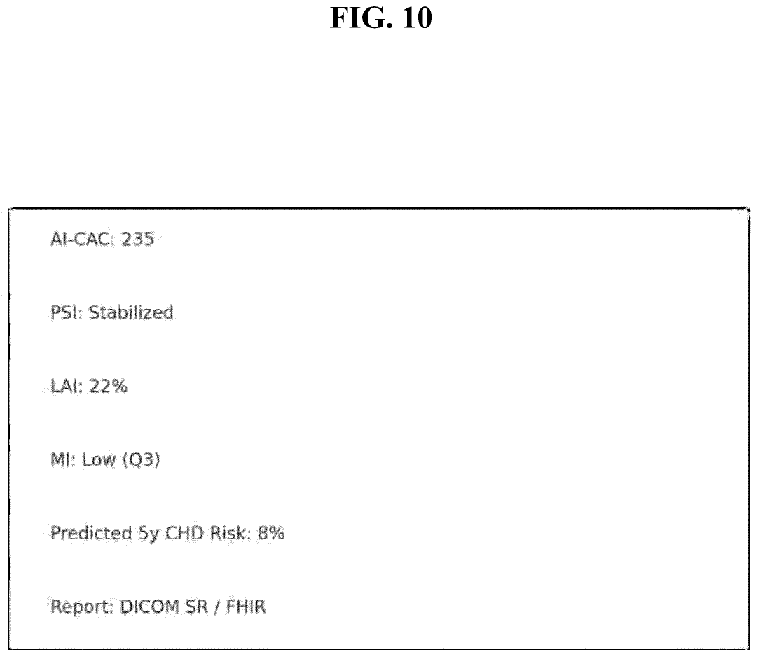

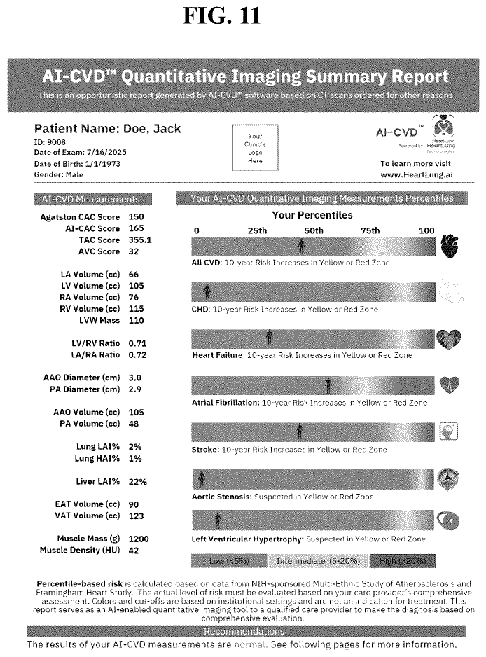

As shown in FIG. 10, outputs AI-CAC, PSI, LAI, and MI are concatenated into a feature vector F. A predictive model—for example a gradient-boosted ensemble or survival neural network—computes probabilities of events including coronary heart disease, heart failure, atrial fibrillation, stroke, chronic obstructive pulmonary disease, diabetes mellitus, and all-cause mortality (FIG. 11).

### Structured Reporting and Visualization

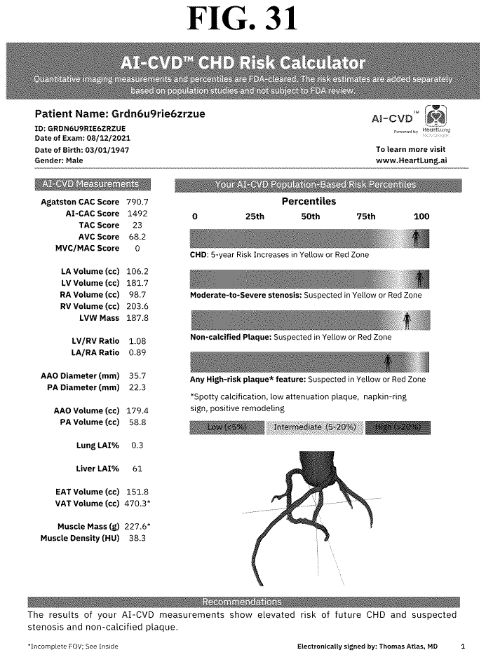

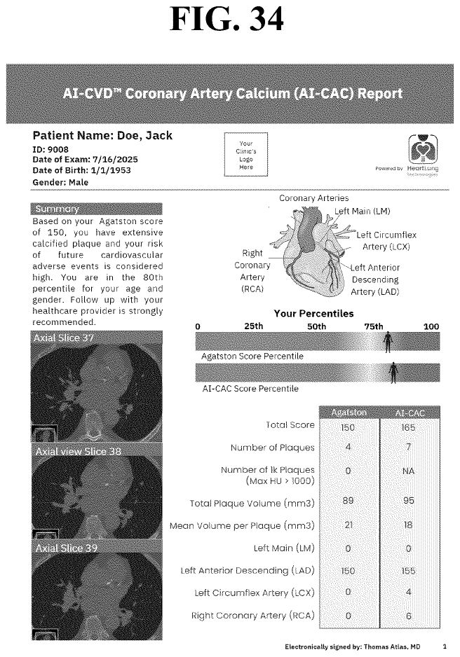

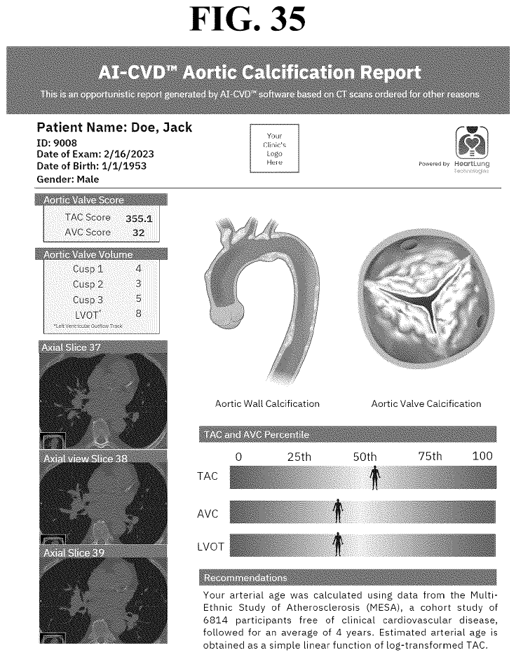

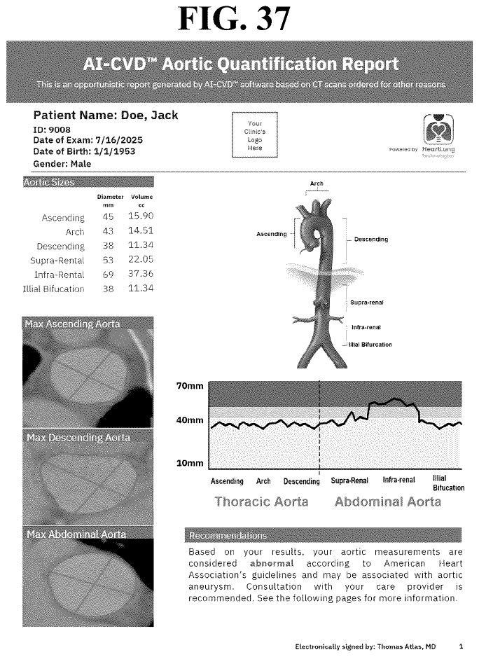

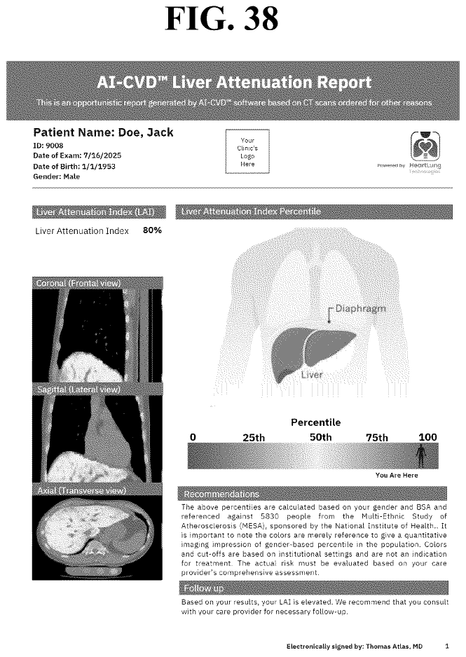

The system generates a structured report containing global and per-vessel AI-CAC scores, PSI classification, LAI, MI, and composite risk estimates. Reports are exported as DICOM Structured Reports or HL7 FHIR resources for electronic-health-record integration. Example visual layout is shown in FIGS. 11 and 31-41.

### System Implementation

Hardware comprises one or more processors, system memory storing executable instructions, and optional graphics processing units (GPUs). Software modules are containerized microservices: segmentation, calibration, plaque analytics, longitudinal matching, and reporting. Modules communicate via APIs, enabling scalable deployment on local servers or cloud infrastructure.

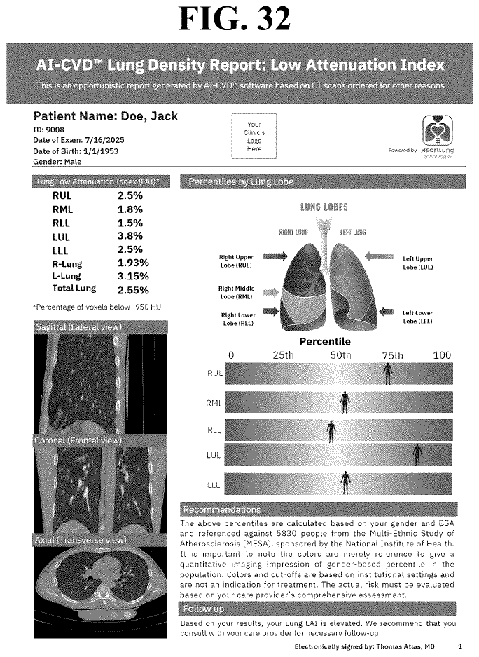

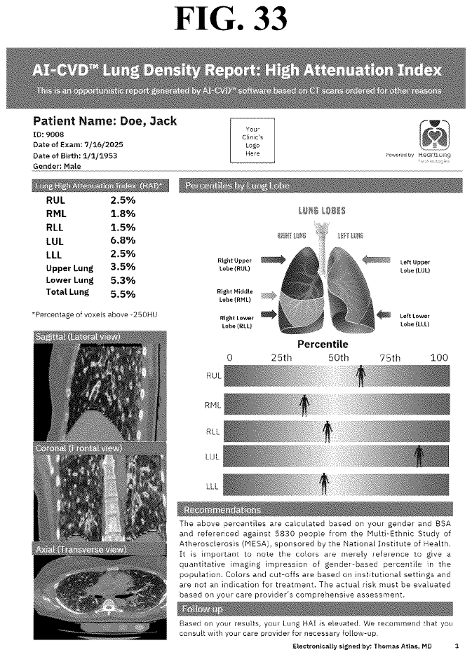

### Extended Cardiopulmonary and Metabolic Characterization (FIG. 32-33) AI-Emphysema Module

A lung-parenchymal segmentation network isolates lung fields and computes the percentage of voxels<−950 HU, yielding a Low-Attenuation Area (LAA %). Regional distribution is mapped by lobe. Higher LAA % correlates with COPD severity and predicts atrial-fibrillation and heart-failure risk independent of CAC.

### AI-Aortic and AI-Pulmonary Modules

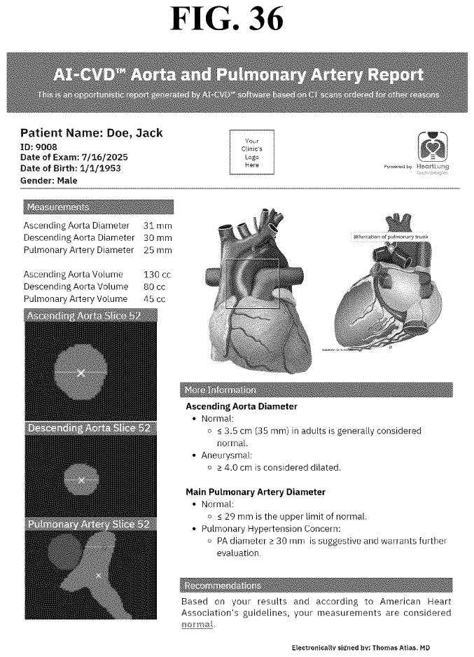

Anatomical models extract the ascending and descending aorta and the main pulmonary artery. The system computes diameters, area ratios (Ao/PA), and detects aortic calcification (TAC) by intensity and morphology. Enlarged Ao or PA diameters indicate hypertension or pulmonary hypertension (FIG. 36).

### AI-Valvular Calcification Module

Using cardiac landmarks, the algorithm segments the aortic and mitral annuli and quantifies calcium burden within +3 mm. Valvular calcium scores correlate with stenosis severity and atrial-fibrillation risk (35).

### AI-Pericardial and Visceral Fat Module

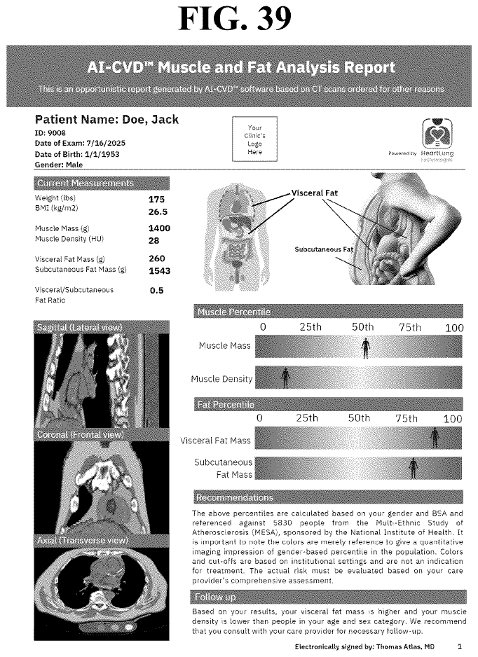

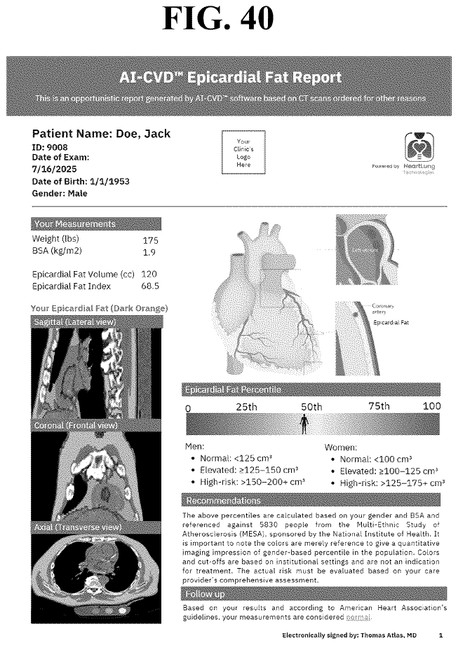

A pericardial-surface model delineates the epicardial contour. Fat voxels (−190 to −30 HU) within this space are summed to compute pericardial-fat volume and mean density. Optional visceral-fat compartments are segmented below the diaphragm. These metrics reflect inflammatory and metabolic load (FIGS. 39 and 40).

### AI-BMD Module (Bone Mineral Density)

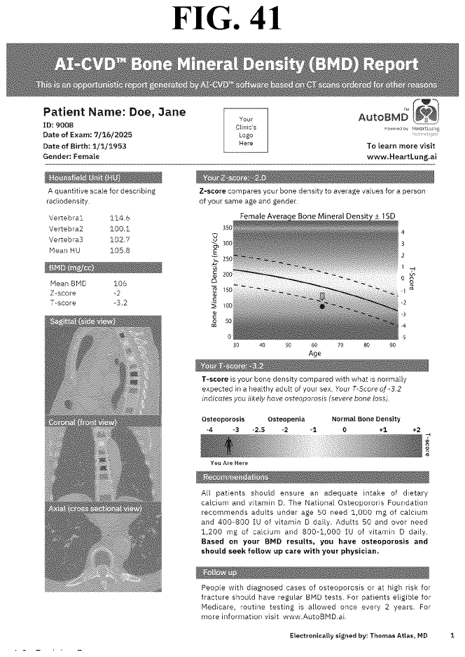

Thoracic Vertebral Bodies are segmented, and mean attenuation values are converted to equivalent T-scores using calibration curves derived from quantitative CT phantoms. The derived BMD is used to predict osteoporosis and can increase cardiovascular mortality (FIG. 41).

### Integrated Risk Model

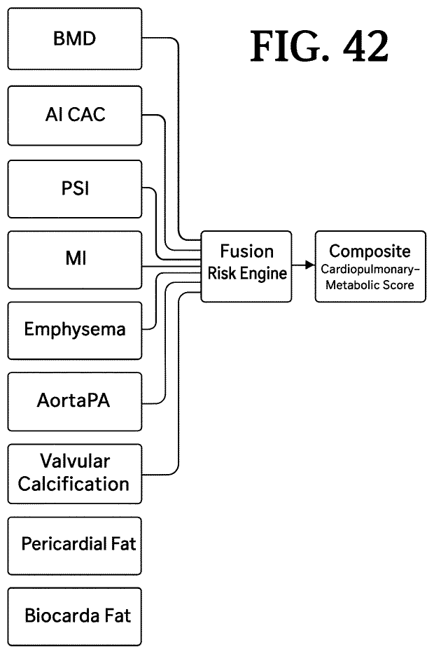

All module outputs {AI-CAC, PSI, LAI, MI, Emphysema, Aorta/PA, Valvular, Pericardial-Fat, BMD} feed a fusion-network (1000) producing a composite cardiopulmonary-metabolic risk score (1002) as shown in FIG. 42. The model employs gradient-boosted or deep-learning fusion layers trained on longitudinal cohort data.

### Advantages of Extended Module

(1) Single CT dataset→multi-organ phenotyping. (2) Improved prediction of cardiovascular, pulmonary, and metabolic outcomes. (3) Population-level opportunistic screening without added dose or cost.

### EXAMPLES

The following examples are presented to illustrate the present invention and to assist one of ordinary skill in making and using the same. The examples are not intended in any way to otherwise limit the scope of the invention.

### Example 1

### Detection of Sub-Threshold Plaques

In a MESA dataset case previously labeled CAC=0 by conventional scoring, AI-CAC detected three small plaques (RDU equivalent≈120 HU) within the proximal LAD, yielding a score of 18 and correctly predicting incident CHD within 5 years.

### Example 2

### Therapy Response Analysis

Among statin-treated participants, plaque density increased 12% while area decreased 3%, generating PSI=+0.27 (classified as stabilized). Untreated subjects showed area ↑ 15%, density ↑ 2%, PSI=−0.19 (progressive).

### Example 3

### Opportunistic Multi-Organ Screening

In a lung-cancer-screening cohort, combined AI-CAC+LAI+MI predicted incident CVD with C-index 0.84 versus 0.69 for CAC alone, demonstrating additive value of hepatic and muscular biomarkers.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions.

The descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. This invention significantly advances preventive cardiology as well as preventive medicine as a whole by empowering physicians to employ CT-based multi-disease early detection and primary prevention.

We claim:

1. A computer-implemented method for quantitatively analyzing non-contrast computed tomography (CT) images, the method comprising:
receiving, by one or more processors, a volumetric non-contrast CT dataset of a subject;
automatically segmenting a coronary artery volume using a trained three-dimensional neural network comprising convolutional and attention layers configured to distinguish arterial structures from adjacent tissues based on spatial continuity and intensity gradients;
transforming voxel intensities to relative density units (RDU) by applying a calibration function derived from at least one in-scan reference region and an adaptive noise estimator, wherein the calibration function reduces scanner-specific variation;
applying an anisotropic near-neighbor spatial filter parameterized by the measured noise to suppress isolated pixels while preserving plaque boundaries;
identifying connected voxel clusters within the segmented arteries whose calibrated intensities exceed a context-adaptive threshold, wherein each voxel cluster represents a candidate calcified plaque;
computing, for each plaque, quantitative descriptors including centroid, three-dimensional volume, mean and percentile RDUs, and intra-plaque density heterogeneity;
generating, by an AI model trained with co-registered contrast-enhanced coronary CT angiography datasets, a plaque stability index (PSI) representing a nonlinear function of per-plaque area-density trajectory across serial scans; and
outputting a structured data record including the PSI, calibrated per-plaque descriptors, and a report suitable for integration into an electronic health record.

2. The method of claim 1, wherein the calibration function uses a subcutaneous fat reference or a 100 HU phantom, and dynamically selects weighting based on signal-to-noise ratio.

3. The method of claim 1, wherein the trained three-dimensional neural network employs a multi-resolution encoder-decoder architecture with residual connections and a vessel-aware attention mechanism trained on paired non-contrast and contrast-enhanced volumes.

4. The method of claim 1, further comprising a step of performing deformable registration between temporally distinct CT datasets of the subject to establish plaque correspondence and calculate Δarea and Δdensity vectors per plaque as well as per each segment of coronary arteries.

5. The method of claim 1, further comprising a step of adjusting an intensity threshold for plaque detection using a learned regression model to predict calcium likelihood based on local noise, voxel size, and RDU histogram shape.

6. The method of claim 1, further comprising a step of classifying therapy response as stabilized when plaque density increases by at least 10% while area change is below 5%, and progressive when area increases beyond 10% irrespective of density trend.

7. The method of claim 1, further comprising a step of concurrently segmenting liver and thoracic muscle regions from the same CT dataset and computing (i) a liver attenuation index (LAI) as the fraction of hepatic voxels <40 HU within [−30, 70] HU, and (ii) a myosteatosis index (MI) as the mean attenuation within the paraspinal muscle mask; wherein the PSI, LAI, and MI are combined within a gradient-boosting or neural network model to predict cardiometabolic risk outcomes.

8. The method of claim 1, wherein the structured data record further comprises per-vessel AI-CAC burden scores for left main (LM), left anterior descending (LAD), left circumflex (LCX), and right coronary artery (RCA).

9. The method of claim 1, further comprising a step of estimating a calibration uncertainty metric derived from noise variance and HU spread to compute confidence intervals for each plaque measurement.

10. The method of claim 1, wherein the plaque stability index (PSI) is calculated using a recurrent neural network trained on longitudinal plaque features to model nonlinear temporal changes in plaque density and area.

11. The method of claim 1, further comprising a step of applying an adaptive histogram equalization before the segmenting step to normalize contrast between bone, fat, and soft tissues.

12. The method of claim 1, wherein the AI model applies a vessel-specific normalization layer that scales intensities according to anatomical segment priors.

13. The method of claim 1, further comprising a step of generating a longitudinal trajectory visualization showing plaque area and density changes over multiple CT acquisitions for the same patient.

14. The method of claim 1, wherein the structured data record is encoded in a DICOM Structured Reporting (SR) or HL7 FHIR format for integration with a clinical information system.

15. A system for opportunistic cardiometabolic analysis of non-contrast CT data comprising:
a processor configured to execute neural networks for segmentation, calibration, and plaque characterization modules;
a memory storing trained model weights, calibration parameters, and temporal alignment algorithms;
a noise-adaptive filtering module configured to apply anisotropic smoothing based on measured image variance;
a feature extraction module configured to compute plaque-level descriptors and longitudinal stability indices; and
an output interface generating a structured clinical report including AI-CAC burden, plaque stability index, liver attenuation index, and myosteatosis index.

16. The system of claim 15, wherein the neural networks include both segmentation and temporal alignment subnetworks coupled by a shared feature encoder.

17. The system of claim 15, further comprising a training subsystem configured to update calibration parameters using cross-scanner harmonization loss to minimize HU drift across different CT models.

18. The system of claim 15, wherein the system integrates outputs from modules that jointly predict risks for coronary heart disease, heart failure, atrial fibrillation, stroke, COPD, diabetes, and mortality.

19. The system of claim 15, further comprising a non-transitory computer-readable medium storing instructions which, when executed by one or more processors, cause the processors to perform the method of any of claims 1 through 14.

20. The system of claim 15, wherein each of the segmentation, calibration, and plaque characterization modules are containerized microservices executable independently or in distributed computing environments, thereby enabling scalable deployment for population-level screening.

21. The system of claim 15, further comprising a lung-parenchymal analysis module configured to segment lung fields and compute an emphysema index based on the proportion of voxels below −950 HU.

22. The system of claim 15, further comprising an analyzer configured to quantify diameters of the ascending and descending aorta and the main pulmonary artery and computes an aorta-to-pulmonary ratio and aortic-calcification burden.

23. The system of claim 22, wherein the analyzer configured to further detect and quantify valvular calcification within aortic or mitral valve regions to generate a valvular-calcification score.

24. The system of claim 15, further comprising a pericardial-fat quantification module configured to delineate the pericardial boundary and compute pericardial-fat volume and density.

25. The system of claim 15, further comprising a bone-mineral density that is determined from thoracic-vertebral attenuation values and converted to T-scores using calibration references.

26. The system of claim 15, wherein the risk engine integrates parameters from claims 21-25 with AI-CAC, PSI, LAI, and MI to generate a composite cardiopulmonary-metabolic risk index and structured report.

27. The system of claim 15, further comprising an event-specific artificial-intelligence model that is trained to predict one or more outcomes selected from heart failure, atrial fibrillation, coronary heart disease, stroke, other cardiovascular events, and all-cause mortality.

28. The system of claim 27, wherein the event-specific artificial-intelligence model accepts features derived from contrast-enhanced or non-contrast, ECG-gated or non-gated CT scans including AI-CAC, PSI, LAI, MI, emphysema, aortic and pulmonary dimensions, valvular calcification, pericardial fat, and bone mineral density.

29. The system of claim 15, further comprising a predictive engine that employs one or more survival-analysis architectures selected from random survival forest, Cox proportional-hazards model, gradient-boosted survival tree, and deep neural survival network.

30. The system of claim 29, wherein the predictive engine generates individualized survival curves and cumulative-incidence functions for each predicted event and displays them as a multi-event risk dashboard.

31. The system of claim 27, wherein the event-specific artificial-intelligence model for coronary heart disease (CHD) can detect obstructive coronary artery disease (stenosis>50%), ischemia, non-calcified plaques, and plaques with high risk (vulnerable plaque) features in non-contrast CT scans.

32. The system of claim 27, wherein the event-specific artificial-intelligence model is retrained by federated-learning procedures across distributed databases without sharing raw patient data.

33. The system of claim 27, wherein outputs from the event-specific artificial-intelligence model is combined by a fusion risk engine to produce a composite cardiopulmonary-metabolic risk score.

34. The system of claim 15, wherein the system automatically recommends preventive therapy or follow-up imaging based on predicted event-specific risk thresholds.
